(19) United States
(12) Patent Application Publication
(10) Pub. No.: US 2011/0028819 A1
(43) Pub. Date: Feb. 3, 2011

(54) MEDICAL SENSOR DEVICE

(75) Inventors: Marcus EGER, Lubeck (DE); Thomas HANDZSUJ, Lubeck (DE); Hans-Ullrich HANSMANN, Barnitz (DE)

Correspondence Address: MCGLEW & TUTTLE, PC, P.O. BOX 9227, SCARBOROUGH STATION, SCARBOROUGH, NY, 10510-9227, US

(73) Assignee: Drager Medical AG & Co. KG, Lubeck (DE)

(21) Appl. No.: 12/841,454

(22) Filed: Jul. 22, 2010

(30) Foreign Application Priority Data
Jul. 28, 2009 (DE) 10 2009 035 018.7

(51) Int. Cl. A61B 5/04

(52) U.S. Cl. 600/372

(57) ABSTRACT

Electromyographic and mechanomyographic parameters of a patient are detected accurately with few artifacts in a medical sensor device (11) for a patient. The medical sensor device (11) includes an electrode (12) for detecting an electric voltage on a body surface of the patient, a holding element (10) with preferably at least one transmission means for transmitting or conducting signals or electric currents, at least one mechanical connection means (14) for the detachable mechanical connection of electrode (12) with holding element (10), and at least one electric connection means (15) for the detachable electric connection of electrode (12) with holding element (10). the holding element (10) comprising at least one sensor for detecting at least one medical parameter of the patient.

### CROSS REFERENCE TO RELATED APPLICATIONS

This application claims the benefit of priority under 35 U.S.C. §119 of German Patent Application 10 2009 035 018.7 filed Jul. 28, 2009, the entire contents of which are incorporated herein by reference.

### FIELD OF THE INVENTION

The present invention pertains to a medical sensor device for a patient including an electrode for detecting an electric voltage on a body surface of the patient, a holding element with preferably at least one transmission means for transmitting or conducting signals or electric currents, at least one, especially detachable mechanical connection means for the detachable mechanical connection of the electrode with the holding element and at least one, and an especially detachable electric connection means for the detachable electric connection of electrode with the holding element.

### BACKGROUND OF THE INVENTION

Sensors are used in medical engineering to detect medical parameters on the skin of patients. The sensors record medical parameters as invasive or noninvasive sensors and pass these on in the form of signals to analyzing units. Electromyographic sensors are used to detect the electric activity of muscles. Electric potential differences, which result from the muscle activity, are detected on the skin surface by at least two sensors. For example, electrodes are used as electromyographic sensors. The parameters detected by electromyographic sensors are also used to control respirators.

The combined detection of parameters by means of electromyographic sensors, i.e., an electromyogram, and the detection of parameters by means of mechanomyographic sensors, i.e., a mechanomyogram (MMG), is especially suitable for assessing muscle efficiency or muscle fatigue. The electromyogram (sEMG) and mechanomyogram (MMG) can well describe muscle efficiency or muscle fatigue because the electromyogram describes the electric activation of the muscle and the mechanomyogram is an indicator of the force exerted by the muscle. Such information on the respiratory muscles is meaningful and valuable especially in connection with patients connected to respirators, especially during the weaning of artificially respirated patients.

Electrodes, which have a contact and adhesive surface for application on the skin surface and, furthermore, a chamber with a gel are used, in general, as electromyographic sensors. The gel is electrically conductive and can be used as a result to detect an electric potential on the skin surface. The electrode with the contact and adhesive surface as well as with the chamber is, in general, a disposable electrode, which is replaced after each use on the patient. The electrode is provided with a mandrel, which is clipped onto a holding element. There is a clip connection between the electrode and the holding element, which connection also has an electric connection for passing on the electric potential, besides the mechanical connection. A cable for passing on the electric current, which was detected on the surface of the patient with the gel, is arranged at the holding element. The holding element is used multiple times. Even though such a medical sensor device does have the advantage that only the component lying directly on the body, namely, the disposable electrode, is replaced and the holding element is used multiple times, so that only the disposable electrode must be exchanged or replaced, it is disadvantageously impossible to detect a mechanomyogram with such a medical sensor device by means of a mechanomyographic sensor.

EP 1 900 323 A1 shows a medical electrode for being adhered to the skin surface of a test subject, with a carrier adhering to the skin and with a holding element for at least one electrically conductive connection piece. The connection piece is covered on the skin side with electrically conductive gel, especially a sponge. A skin-side gap between the holding element and the electrically conductive connection piece is closed by a preferably ring-shaped sealing element.

DE 10 2007 021 960 A1 shows an electrode for detecting electric signals on a body surface with a carrier layer facing away from the body and with an electrically conductive adhesive layer, which is arranged on the carrier layer and faces the body. The carrier layer has a magnetizable coupling means for coupling the carrier layer with an electrode holder by means of magnetic force.

A medical sound sensor for detecting heart sound and/or lung sounds is known from DE 10 2007 001 921 B3. A sensor housing with an opening is spanned over with an electric membrane. A piezoelectric vibrating element, which is arranged in a damping oil, is used to convert mechanical vibrations of the membrane into electric signals.

### SUMMARY OF THE INVENTION

The object of the present invention is therefore to make available a medical sensor device and a medical sensor system, in which electromyographic and mechanomyographic parameters of a patient can be reliably detected.

This object is accomplished with a medical sensor device for a patient, comprising an electrode for detecting an electric voltage on the body surface of a patient, a holding element with preferably at least one transmission means for transmitting or conducting signals or electric currents, at least one mechanical connection means for the detachable mechanical connection of the electrode to the holding element, at least one electric connection means for the detachable electric connection of the electrode to the holding element, wherein the holding element comprises at least one sensor for detecting at least one medical parameter of the patient.

In particular, the at least one sensor for detecting at least one mechanical parameter of the patient is integrated in the holding element and/or is rigidly connected to the holding element and/or is detachably or nondetachably connected to the holding element.

Besides an electromyogram, other parameters of the patient, especially a mechanomyogram, can thus advantageously also be detected in a medical sensor device with a holding element as a reusable component and with an electrode as a disposable electrode.

The at least one sensor is, in particular, a mechanomyographic sensor.

In another embodiment, the at least one sensor is an acceleration sensor and/or a microphone.

In an additional embodiment, the at least one sensor is a piezoelectric contact sensor. The piezoelectric contact sensor is, in general, an acceleration sensor. The accelerations acting on a piezo element lead, based on the mass of the piezo element, to a force, which is detected by the piezo element. The at least one mechanical connection means is preferably designed such that the coefficient of friction equals at least 0.05 or 0.1 and preferably 0.15 or 0.25 at right angles to a contact surface of the electrode in the embodiment of a frictionally engaged and/or positive-locking connection between the holding element and the electrode, especially between a contact surface of the holding element and a contact surface of the electrode, and/or the prestressing force equals at least 0.5 N or 1 N and preferably at least 2 N, 3 N or 10 N in an embodiment as a non-positive connection on the contact surface. The at least one mechanomyographic sensor is arranged at the holding element. The patient performs, especially during breathing, motions on the body surface, which propagate to the medical sensor device. To prevent the parameters detected by the mechanomyographic sensors from being distorted because of a mechanical clearance between the holding element and the electrode, it is necessary that no mechanical clearance or no relative motion take place between the holding element and the electrode during such motions. The friction values and prestressing forces indicated are therefore necessary.

In one variant, the at least one mechanical connection means is designed such that the static friction between the holding element and the electrode, especially between the contact surface of the holding element and the contact surface of the electrode, at right angles to a contact surface of the electrode equals at least 0.05 N, 0.1 N or 0.2 N, preferably at least 0.3 N, 0.5 N, 1 N, 2 N, 5 N or 10 N.

A clip or locking connection, especially a detachable clip or locking connection, can be preferably established by means of the at least one mechanical connection means and/or a positive-locking and/or nonpositive connection, especially a detachable positive-locking and/or nonpositive connection, can be established by means of the at least one mechanical connection means.

In another embodiment, the at least one mechanical connection means comprises a mandrel arranged in a recess, and the mandrel is preferably formed at the electrode and the recess at the holding element.

In particular, the at least one mechanical connection means is prestressed by means of an elastic element, especially a spring or an O-ring. The elastic ring brings about a prestress at the mechanical connection means, e.g., at a disk and/or a mandrel, so that no mechanical clearance will essentially occur as a result when mechanical loads occur between the holding element and the electrode, so that not even motions on the patient's body surface will as a result distort the measurements of the at least one sensor, especially of the at least one mechanomyographic sensor.

The prestress of the at least one mechanical connection means is brought about by a tilting of the holding element in relation to the disk and/or the mandrel. As a result, the mechanical degrees of freedom caused by the relative motions between the holding element and the electrode, which do not affect the functionality of the electric signal tapping in case of an only electrically conductive clip or locking connection, are compensated, so that there also will be a conductivity of mechanical and acoustic pulses, of acceleration signals from the skin surface of a patient through the connection means to the at least one mechanomyographic sensor arranged in the holding element in case of the application of the present invention. The prestress brought about by the tilting results in a nonpositive connection between the holding element by means of the mandrel and the electrode. In addition or as an alternative to the nonpositive connection, a frictionally engaged and/or positive-locking connection can also be achieved by the dimensioning and tolerance zone selection of the dimensions of the holding element preferably designed as a clip or locking connection and the mandrel arranged on the electrode in case of connecting the holding element to the mandrel. For this application on the human body and the interference factors and influencing variables prevailing there, a value of 1 N to 20 N has been determined for a nonpositive connection in measuring experiments for an adequate and robust design of the connection means with reliable transmission of the mechanomyographic signals from the skin surface to the sensor. Coefficients of friction of at least 0.2 were determined for a frictionally engaged and/or positive-locking connection without supporting combination by a nonpositive connection component in the connection means. The coefficients of friction are determined essentially by the elasticity of the materials involved in the frictionally engaged and/or positive-locking connection and the surface quality, i.e., roughness of the elements of the frictionally engaged and/or positive-locking connection, i.e., especially of the mandrel, electrode and holding element. It should be borne in mind in this connection that the surface quality must be such in reusable elements that the residual microorganism count left behind after cleaning by means of disinfection and/or autoclave treatment must meet the hygienic requirements imposed in routine clinical practice. A corresponding material and a corresponding surface quality, which takes the hygienic aspects into account, must therefore be selected in case of a combination of disposable elements, in this case the electrode, with reusable elements, in this case the holding element. This means that for a frictionally engaged and/or positive-locking connection, the holding element should be made of elements consisting of an elastic material with a rather smooth surface, whereas the electrode and the mandrel as disposable elements can be roughened up and structured. Coefficients of friction of about 0.1 are obtained for the positive-locking connection in case of a combination of a nonpositive connection and a frictionally engaged and/or positive-locking connection for the connection element, and values of about 10 N are obtained for the frictionally engaged connection of a prestress brought about by tilting, for example, by means of a mechanical spring arranged in the holding element. This combination offers the advantage that the user can easily and reliably make and undo the connection. Excessively high coefficients of friction of >0.3 and prestresses of >10 N generated with the making of the connection require large dimensions for the electrodes to ensure that the distribution of forces during the making of the connection can be distributed over a large skin surface and is not therefore painful for the patient. Large dimensions of the electrodes are not practical in routine clinical practice, and the proposed combination of nonpositive connection and positive-locking connection thus represents a meaningful solution from a medical and measuring technical point of view and from the viewpoint of handling.

In another embodiment, the electrode comprises a chamber with gel and/or the contact surface is designed as an adhesive surface.

In an additional variant, the electrode is a disposable electrode and/or the holding element can be reused several times. The electrode as an inexpensive disposable electrode is replaced after each use and cannot be reused. The holding element with transmission means, e.g., a cable, is used for a rather long time. As a result, the holding element does not need to be replaced and only the inexpensive disposable electrode is to be replaced after use or measurement.

In another variant, the at least one transmission means for transmitting or conducting signals or electric currents is a cable and/or a signal processing and transmitting unit and preferably a receiver.

To transmit mechanical and acoustic pulses, especially acceleration signals, within the variants of the medical sensor device according to the present invention described according to the present invention, it is necessary for a reliable and reproducible transmission channel from the skin surface of a patient through the connection means to the at least one mechanomyographic sensor arranged in the holding element that the signal-attenuating properties of the masses and materials involved and of the interfaces between the elements and materials involved of the transmission channel be adapted such that the frequency components necessary for the analysis will not be damped in the signal by the vibration time constant of the entire arrangement so strongly that the signals of the mechanomyographic sensor will not have any measuring technical information value of the mechanomyogram any more in respect to the muscle activity. The marked frequencies of the mechanomyogram are in the range from a few Hz to 50 Hz. A limiting frequency of 200 Hz is necessary in a technical embodiment for a qualitatively good analyzability for the series connection of the elements involved, namely, muscle, fat and human skin, gel arranged at the electrode in a chamber, electrode with mandrel attachment means and mandrel, holding element with mechanomyographic sensor and the connection cable, which said series connection acts as a low-pass filter. For the case of a nonattenuated vibration, an effective natural frequency Ω is determined according to the following formula with the hypothesis of a single-mass oscillator, in which c is the spring rate and m is the mass of the elements arranged in the series connection.

$$Ω = \sqrt{\frac{c}{m}}$$

The size, thickness and material properties, essentially the modulus of elasticity, of the material are expressed in the spring constants.

Measurements and experiments have shown that the described series connection of the elements summarily has a mass of less than 20 g, which results in a low-pass natural frequency of about 400 Hz in cooperation with the material properties. Thus, qualitatively good analyzability is guaranteed with the medical sensor device according to the present invention.

A sensor system according to the present invention has at least two medical sensor devices, comprising an electrode for detecting an electric voltage on the body surface of a patient, a holding element with at least one transmission means for transmitting or conducting signals or electric currents, at least one mechanical connection means for the detachable mechanical connection of the electrode to the holding element, at least one electric connection means for the detachable electric connection of the electrode with the holding element, wherein the at least two medical sensor devices are connected to one another by means of the at least one transmission means, e.g., a cable or a signal processing and transmitting unit and/or a receiver, wherein at least one medical sensor device of the sensor system according to the present invention is designed according to a medical sensor device described in this patent application.

At least two sensor devices, e.g., three to six medical sensor devices, are, in general, connected to one another by means of a cable and thus form a sensor system.

Another sensor system according to the present invention has at least two medical sensor devices, comprising an electrode for detecting an electric voltage on the body surface of a patient, a holding element with at least one transmission means for transmitting or conducting signals or electric currents, at least one mechanical connection means for the detachable mechanical connection of the electrode with the holding element, at least one electric connection means for the detachable electric connection of the electrode with the holding element, wherein the at least two medical sensor devices are connected to one another by means of the at least one transmission means, e.g., a cable or a signal processing and transmitting unit and/or receiver, wherein at least one mechanomyographic sensor is arranged outside the holding element.

In another embodiment, the at least one mechanomyographic is arranged indirectly or directly at the cable or is connected thereto.

Exemplary embodiments of the present invention will be described in more detail below with reference to the attached drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a longitudinal sectional view through a medical sensor device according to the invention;

FIG. 2 is a longitudinal sectional view of a medical sensor system according to the invention;

FIG. 2a is a schematic view of the signal detection, signal processing and data transmission of a medical sensor system according to the invention;

FIG. 3 is a first longitudinal sectional view of the medical sensor device in a first exemplary embodiment according to the invention;

FIG. 3a is a second longitudinal sectional view of the medical sensor device in the first exemplary embodiment;

FIG. 4 is a first longitudinal sectional view of the medical sensor device in a second exemplary embodiment according to the invention;

FIG. 4a is a second longitudinal sectional view of the second exemplary embodiment;

FIG. 4b is a third longitudinal sectional view of the medical sensor device in the second exemplary embodiment;

FIG. 5 is a longitudinal sectional view of the medical sensor device in a third exemplary embodiment according to the invention;

FIG. 6 is a longitudinal sectional view of the medical sensor device in a fourth exemplary embodiment according to the invention;

FIG. 7 is a longitudinal sectional view of the medical sensor device in a fifth exemplary embodiment according to the invention;

FIG. 8 is a longitudinal sectional view of the medical sensor device in a sixth exemplary embodiment according to the invention;

FIG. 9 is a longitudinal sectional view of the medical sensor device in a seventh exemplary embodiment according to the invention;

FIG. 10 is a first signal-vs.-time diagram showing an ECG signal and a breathing signal; and

FIG. 11 is a second signal-vs.-time diagram showing a view of an ECG signal, a MMG signal and a breathing signal.

### DESCRIPTION OF THE PREFERRED EMBODIMENTS

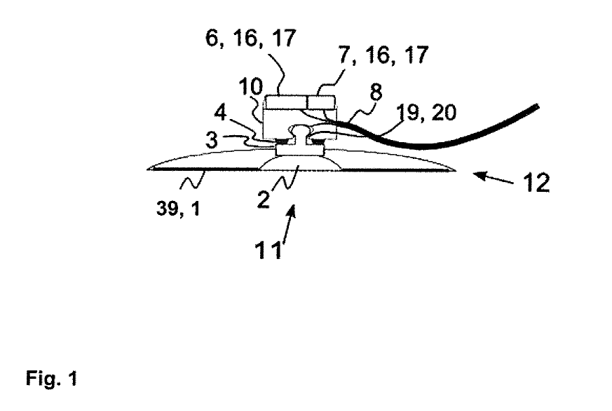

Referring to the drawings in particular, FIG. 1 shows a longitudinal section of a medical sensor device 11 for detecting an electromyogram (sEMG) and a mechanomyogram (MMG). The medical sensor device 11 is used especially in the artificial respiration of patients in order to detect the muscle efficiency and muscle fatigue of the respiratory muscles.

The medical sensor device 11 has a holding element 10 and an electrode 12. The electrode 12 is detachably connected to the holding element 10 by means of a clip and locking connection 19, 20, not shown in detail. Electrode 12 is a disposable electrode and is replaced after each use. A contact surface 39 acting as an adhesive surface 1 and a chamber 2 with a gel are present at the electrode 12. Electrode 12 is attached to the skin surface of the patient by adhesion by means of the contact surface 39 and the adhesive surface 1. To conduct electric potentials or an electric current, chamber 2 is provided with an electrically conductive gel. Furthermore, a mandrel 21, which meshes with a recess 22 of holding element 10, is present at electrode 12. Furthermore, an elastic O-ring 4, which leads to bracing of the clip and locking connection 19, 20, is present between the holding element 10 and electrode 12. Mandrel 21 is formed at a mandrel attachment means 3 of electrode 12. Electrode 12 thus represents an electromyographic sensor. An acceleration sensor 6 and a microphone 7 acting as sensors 16 for detecting a medical parameter, especially as mechanomyographic sensors 17, are arranged at holding element 10. The medical sensor device 11 can thus detect both an electromyogram (sEMG) by means of electrode 12 and a mechanomyogram (MMG) by means of the acceleration sensor 6 and microphone 7.

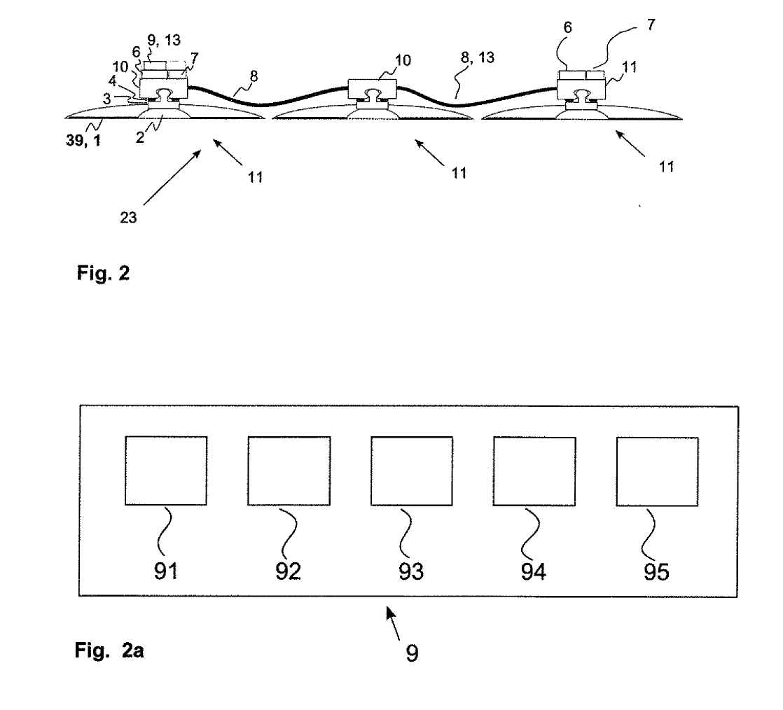

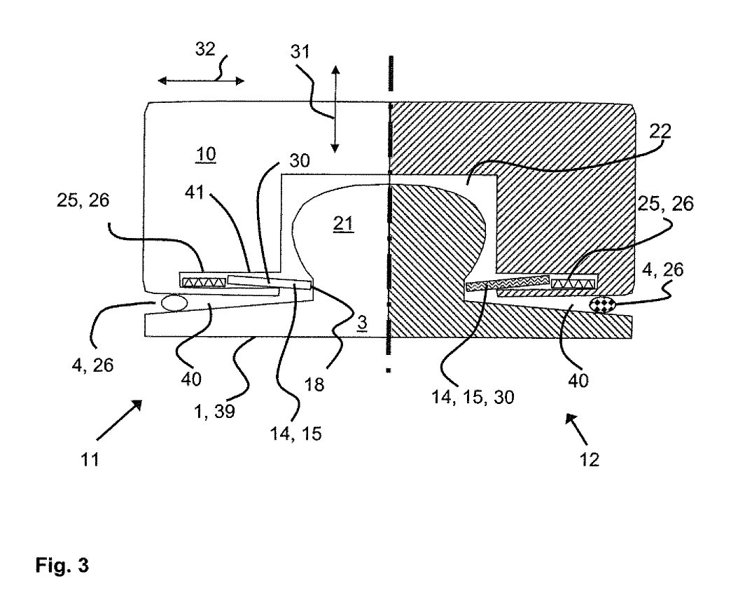

A sensor system 23 shown in FIG. 2 comprises three mechanical sensor devices 11. The medical sensor device 11 shown on the right-hand side of FIG. 2 corresponds to the sensor device 11 shown in FIG. 1. The sensor device 11 shown on the left-hand side of FIG. 2 additionally comprises a signal processing and transmitting unit 9 and preferably a receiver for transmitting data and/or signals. The signal processing and transmitter/receiver unit 9 thus represents, besides cable 8, a transmission means 13 for transmitting or conducting signals or electric currents. The three sensor devices 11 are connected to one another by means of cable 8, and the data detected by the three sensor devices 11 are transmitted to an analyzing unit (not shown). The sensor devices 11 according to FIG. 2 also have, furthermore, a battery, not shown, suitably designed for supplying the acceleration sensor 9 [sic-Tr.Ed.], microphone 7 and signal processing and transmitter/receiver unit 9 with electricity. In another sensor system 23, not shown, the sensor devices 11 have no signal processing and transmitting unit 9 and the sensor devices 11 are connected to the analyzing unit by means of cable 8 as a transmission means for transmitting or conducting signals or currents 13, so that both the transmission of data and signals and supply with electricity can be performed by means of cable 8.

FIG. 2a shows a schematic view of the components of the signal processing and transmitter/receiver unit 9. The signal processing and transmitting unit 9 comprises an amplifier 91, an element for analog-digital conversion 92, an element for coding 93, a unit for modulation 94 and an HF transmitting and receiving stage 95. Individual components, e.g., the A/D converter 92, coding unit 93 and modulating unit 94, are integrated in the practical embodiment in a microcontroller assembly unit designed as a computing unit.

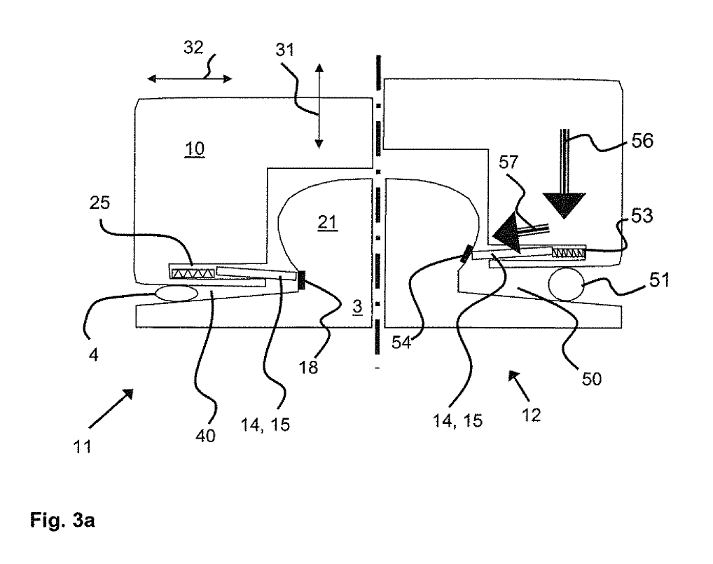

FIG. 3 shows a longitudinal section of the medical sensor device 11 in a first exemplary embodiment. The acceleration sensor 6 and the microphone 7 as well as the adhesive surface 1 or the contact surface 39 and chamber 2 with the gel are not shown in FIG. 3. The view in FIG. 3 is used essentially to describe the first exemplary embodiment of the mechanical connection means 14 and of the electric connection means 15 between holding element 10 and electrode 12. Mandrel 21 is formed at the mandrel attachment means 3 of electrode 12. Mandrel 21 meshes with recess 22 of the holding element 10. An annular groove 41, in which an elastic element 26 designed as a spring 25 and a disk 30 are arranged concentrically, is milled into holding element 10. One end of disk 30 is thus pressed at a contact surface 18 onto a contact surface 18 at mandrel 21 and at the mandrel attachment means 3. As a result, a nonpositive connection becomes established between disk 30 and mandrel 21 or the mandrel attachment means 3.

Mandrel attachment means 3 is designed such that it is electrically conductive to the chamber 2, not shown, with the gel, so that electric current can be sent as a result through the mandrel attachment means 3, disk 30 and spring 25 as well as the holding element 10 and the limiting walls of annular groove 41. Thus, these components represent an electric connection means 15 for electrically connecting electrode 12 to holding element 10. In addition, these components also represent a mechanical connection means 14 because a mechanical connection is established between holding element 10 and electrode 12 due to the nonpositive connection between the contact surfaces 18 at disk 30 and the mandrel attachment means 3 or mandrel 21. An annular gap 40, in which the O-ring 4 is present as another elastic element 26, is present between electrode 12 and holding element 10.

O-ring 4 brings about a bracing of the mechanical connection means 14, so that a nonpositive and/or frictionally engaged [and] positive-locking connection is also established as a result between holding element 10 and electrode 12. As a result, no mechanical clearance develops between the electrode 12 and the holding element 10 due to motions on the skin surface of the patient, so that the parameters detected by the mechanomyographic sensors 17 as an acceleration sensor 6 and microphone 7 are advantageously also not distorted as a result by a mechanical clearance between electrode 12 and holding element 10. Thus, no clearance develops in a direction 31 at right angles to the contact surface 39. Moreover, no mechanical clearance will advantageously develop analogously in a direction 32 in parallel to the contact surface 39 either, because this mechanical connection also has essentially the same mechanical properties in direction 32 because of the bracing and the nonpositive connection by means of the mechanical connection means 14.

The electric potentials detected or electric currents received by electrode 12 are passed on to an analyzing unit (not shown) by means of the cable 8 not shown in FIG. 3. Cable 8 and/or the signal processing and transmitter/receiver unit 9 thus represent the transmission means 13 for transmitting or conducting signals or electric currents (FIGS. 1, 2 and 7 through 9).

FIG. 3a shows a longitudinal section of the medical sensor device 11 in a first exemplary embodiment in a detail view. The view in FIG. 3a is used to describe the functional action of the first exemplary embodiment of the mechanical connection means 14 and of the electric connection means 15 between holding element 10 and electrode 12 with the mandrel 21 according to FIG. 3. Identical elements in FIG. 3a are designated by the same reference numbers as in FIG. 3. Only the elements that are necessary for showing the functional action of the first exemplary embodiment are provided with reference numbers in FIG. 3a. The action of the bracing of the connection is graphically represented by the views on the left- and right-hand sides in a longitudinal section.

A prestressed O-ring 4 as an elastic element 26 (FIG. 3) and a spring 25 in the resting position as another elastic element 26 (FIG. 3) are shown on the left-hand side of FIG. 3a.

An enlarged annular gap 50 with the O-ring 4 in the relaxed state 51 as an elastic element 26 (FIG. 3) and the spring 25 in a more heavily stressed state 53 as another elastic element 26 (FIG. 3) are shown on the right-hand side of FIG. 3a. The stressed spring 53 on the right-hand side is stressed more heavily than spring 25 in the resting position. Due to the bracing in different vertical positions, the mechanical and electric connection means 14, 15 are connected to mandrel 21 via contact surfaces 54, 18, which are especially highlighted in this FIG. 3a, on the right side and on the left side. Due to the shape of mandrel 21, the spring tension of spring 25 is lower on the left-hand side than that of spring 53 on the right-hand side; in cooperation with the stressed O-ring 4 and the spring, the mechanical and electric connection means 14, 15 are pressed here onto the contact surface 18 of mandrel 21. Due to a vertical pressure 56 and due to the interaction of the spring tension of spring 53 and the relaxed O-ring 51, the mechanical and electric connection means 14, 15 are pressed on the right-hand side onto a contact surface 54 of mandrel 54 with an essentially horizontal pressure 57. The degree of bracing changes with increasing vertical pressure 56 and O-ring 51 is prestressed more heavily, the mechanical and electric connection means 14, 15 are pressed by the prestress of spring 53 to the vertical center of mandrel 21 with an essentially horizontal pressure 57 until a nonpositive and/or positive-locking connection will again develop between holding element 10 and electrode 12 in an equilibrium of forces due to the bracing by mandrel 21 and the mechanical connection means 14. To make it possible to bring about bracing via the mechanical connection means 14 on the arrangement comprising the holding element 10, mandrel 21 and electrode 12, a ratio ranging from 1.8:1 to 1.2:1 of the diameter of the O-ring 51 in the relaxed state to the diameter of the prestressed O-ring 4, for which ratio the O-rings 4, 51 are designed by a suitable material composition, has proved to be advantageous in the practical implementation.

Based on this bracing and the nonpositive connection by means of the mechanical connection means 14, no clearance develops in direction 31 at right angles to the contact surface 39 and in direction 32 in parallel to the contact surface 39.

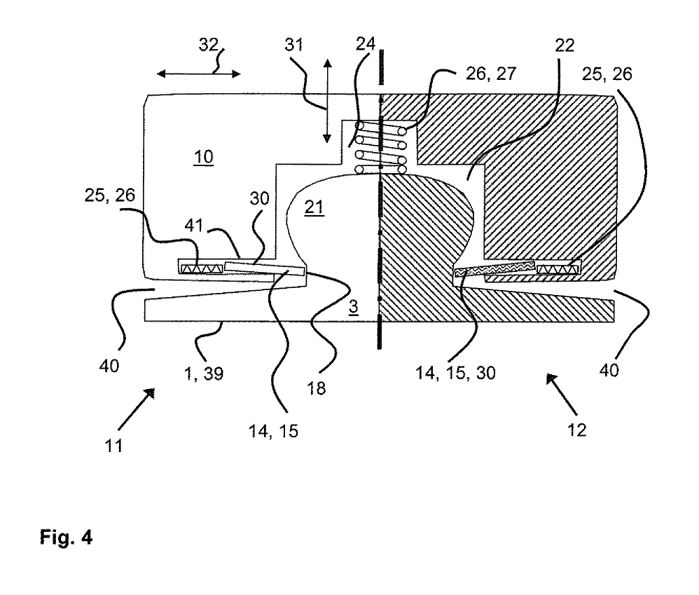

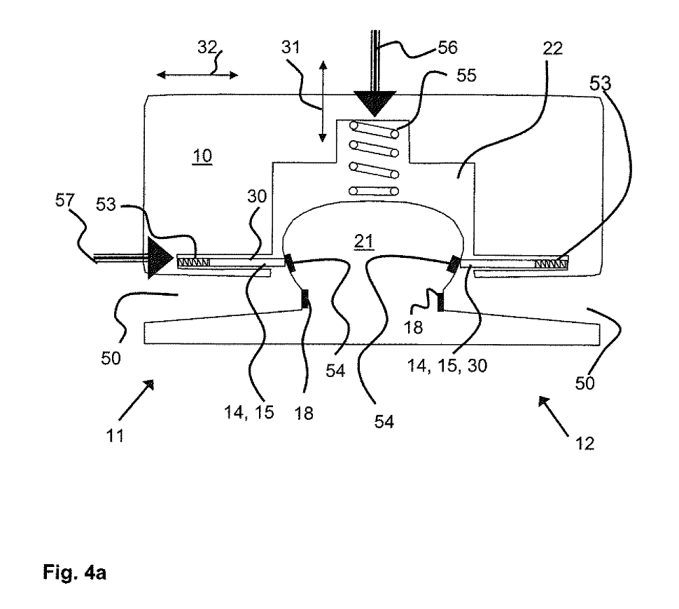

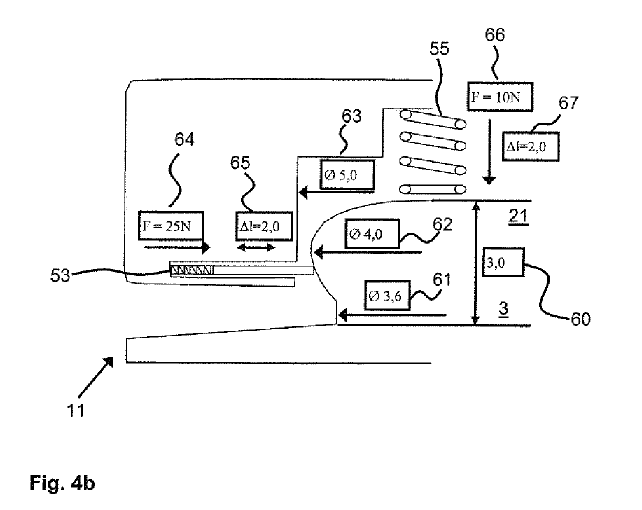

The second exemplary embodiment of the medical sensor device 11 shown in FIG. 4 corresponds essentially to the first exemplary embodiment according to FIG. 3 and differs only in that a mandrel spring 27 is used as an elastic element 26 for bracing instead of O-ring 4. Mandrel spring 27 is arranged in a mandrel spring recess 24 of holding element 10.

The second exemplary embodiment of the medical sensor device 11 according to FIG. 4 is shown in FIG. 4a. Identical elements in FIG. 4a are designated by the same reference numbers as in FIG. 4. Only the elements that are necessary for showing the functional action of the second exemplary embodiment are provided with reference numbers in FIG. 4a. Mandrel spring 27 is shown in a relaxed state 55. The mechanical and electric connection means 14, 15 are connected to mandrel 21 in vertically variable positions of contact surfaces 18, 54, which positions are especially highlighted in this FIG. 4a. Due to the shape of mandrel 21, the lateral springs 53 are shown in a move heavily stressed state. Due to a vertical pressure 56, the mode of bracing changes and the mechanical and electric connection means 14, 15 are pressed by the prestress of the springs 53 to the vertical center of mandrel 21 with an essentially horizontal pressure 57 until a nonpositive and/or positive-locking connection will again become established between holding element 10 and electrode 12 in a balance of forces due to the bracing by mandrel 21 and the mechanical connection means 14. Based on this bracing and the nonpositive connection by means of the mechanical connection means 14, no clearance develops in direction 31 at right angles to the contact surface 39 and in direction 32 in parallel to the contact surface 39.

FIG. 4b shows the second exemplary embodiment of the medical sensor device 11 according to FIG. 4 and FIG. 4a with indication of dimensions, spring travel strokes and spring forces in a halved longitudinal section. Identical elements in FIG. 4b are designated by the same reference numbers as in FIG. 4 and FIG. 4a. Only the elements of FIG. 4 and FIG. 4a that are necessary for showing the dimensions, spring travel strokes and spring forces are shown and provided with reference numbers. Mandrel spring 27 is shown in a relaxed state 55. Mandrel 21 has a height 60 of 3.6 mm, a lower diameter 61 of 3.0 mm at the mandrel attachment and a maximum horizontal diameter 62 of 4.0 mm. Recess 22 in holding element 10 has an internal diameter 63 of 5.0 mm. The lateral spring force 64 of lateral spring 53 equals 25 N with a horizontal spring travel stroke 65 of 2.0 mm. The vertical spring force 66 of mandrel spring 55 equals 10 N with a vertical spring travel stroke 67 of 2.0 mm.

The third exemplary embodiment of the medical sensor device 11 shown in FIG. 5 analogously corresponds to the first exemplary embodiment according to FIG. 3, where only a U-shaped prestressing element 28 is used instead of O-ring 4 for the mechanical bracing of holding element 10 with electrode 12 by the mechanical connection means 14.

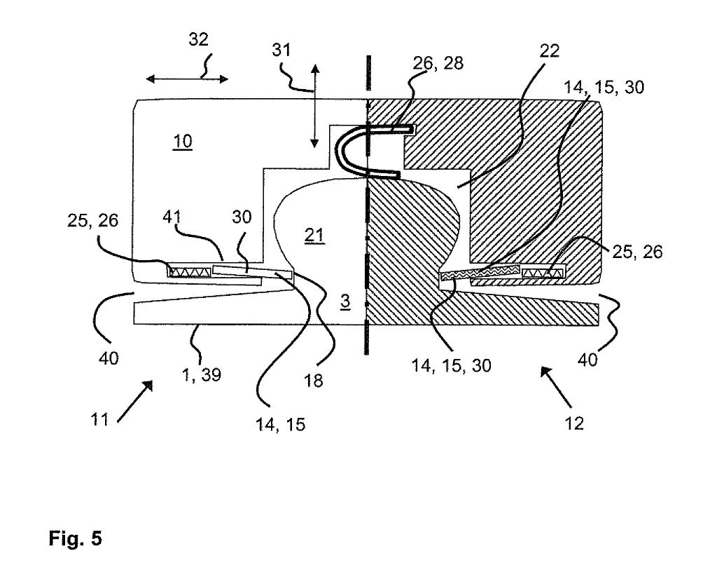

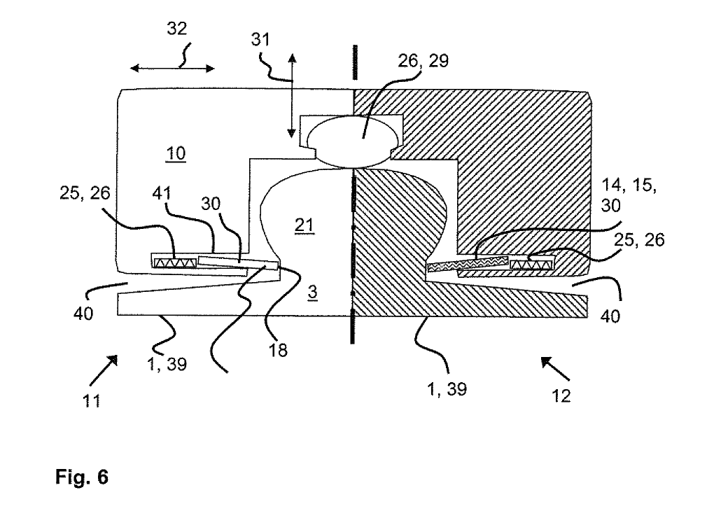

The fourth exemplary embodiment according to FIG. 6 analogously corresponds to the first exemplary embodiment according to FIG. 3, wherein only an elastic pad 29 is used instead of O-ring 4 to brace the mechanical connection means 14.

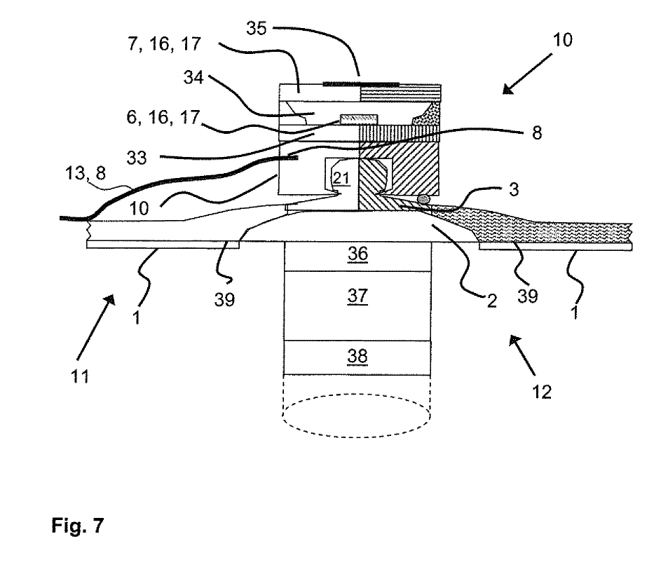

FIG. 7 shows a fifth exemplary embodiment of the medical sensor device 11. The mechanical and electric connection means 14, 15 between holding element 10 and electrode 12 correspond here to the first exemplary embodiment according to FIG. 3 and are essentially not shown in FIG. 7. Electrode 12 lies with the contact surface 39 on the skin 36 of a patient and is connected to the skin 36 by an adhesive connection by means of the adhesive surface 1. Fat 37 and muscle 38 are still present under the skin 36. A printed circuit board 33 for electric circuits (PCB: printed circuit board) is arranged at the holding element 10 made of plastic above recess 22 for the mandrel 21. The acceleration sensor 6 is arranged as a mechanomyographic sensor 17 on the printed circuit board 33. An elastic damping layer 34 is present above the printed circuit board 33 and the microphone 7 with a membrane 35 is present on the damping layer 34. Damping layer 34 is used to damp abrupt motions in order not to distort the measurements by the microphone 7. The data or signals detected by the electrode 12 as an electromyographic sensor and by the acceleration sensor 6 and the microphone 7 as a mechanomyographic sensor 17 are passed on by means of cable 8 as a transmission means 13 for transmitting or conducting signals or electric currents to an analyzing unit.

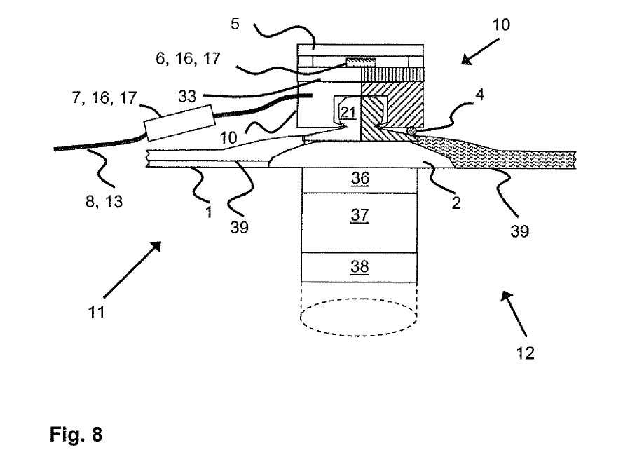

The medical sensor device 11 shown in FIG. 8 in a sixth exemplary embodiment corresponds essentially to the medical sensor device 11 shown in FIG. 7. Essentially only the difference from the fifth exemplary embodiment shown in FIG. 7 will be described below. Microphone 7 is fastened to cable 8 instead of above the damping layer 34. As a result, the sensor device 11 according to FIG. 8 has no damping layer 34 at the holding element 10. The holding element 10 is closed with a cover plate 5 above the acceleration sensor 6.

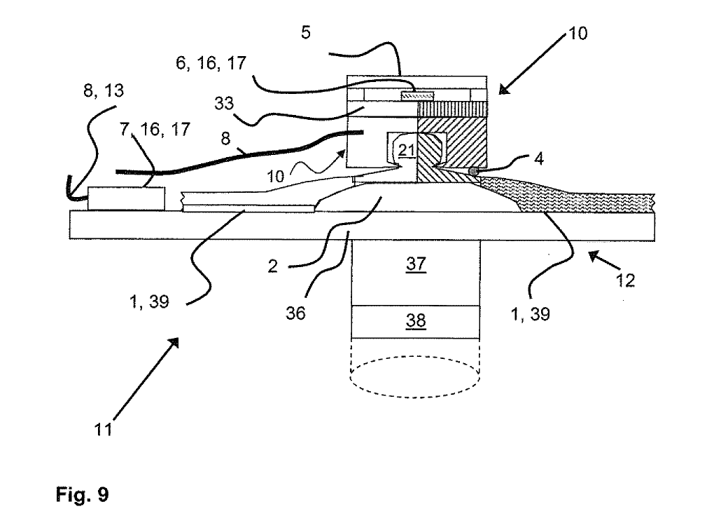

The seventh exemplary embodiment of the medical sensor device 11 shown in FIG. 9 corresponds essentially to the sixth exemplary embodiment shown in FIG. 8. Essentially only the differences from the sixth exemplary embodiment according to FIG. 8 will be described below. Microphone 7 is not fastened directly to the cable 8 but is electrically connected to the analyzing unit by means of a separate cable 8 for the microphone 7 only and is placed or adhered to the skin 36 of the patient. The separate cable 8 is either connected to the analyzing unit indirectly or it opens into the cable 8 for passing on data or signals from the holding element 10.

The medical sensor device 11 preferably consists of plastic. In particular, the holding element 10 in the area of recess 22 and the electrode 12 outside the chamber 2 consist of plastic. Disk 30 preferably consists of electrically conductive metal.

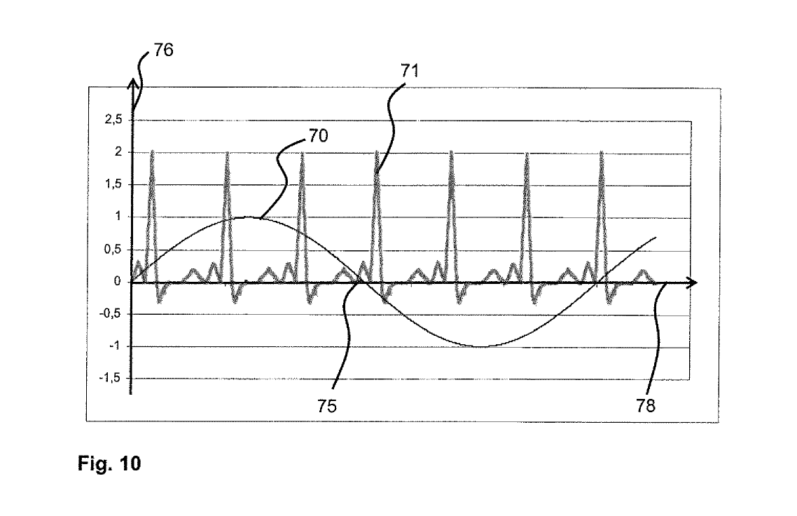

FIG. 10 schematically shows a first signal-vs.-time diagram with a representation of an ECG signal 71 and a breathing signal 70 of a human being, for example, a patient. A plotting of the signal amplitude of the ECG signal 71 on the Y axis is shown in a first range of values 76 from −1.5 mV to +2.5 mV. Furthermore, a representation of a breathing signal 70 in a dimensionless and standardized form is shown, where a value of +1.0 corresponds to inspiration and a value of −1.0 corresponds to an expiration. Changeover times 75 between inspiration and expiration are shown in the breathing signal 70. A time interval with a breath with inspiration and expiration is shown on the time axis 78, this diagram showing seven ECG complexes in the time interval.

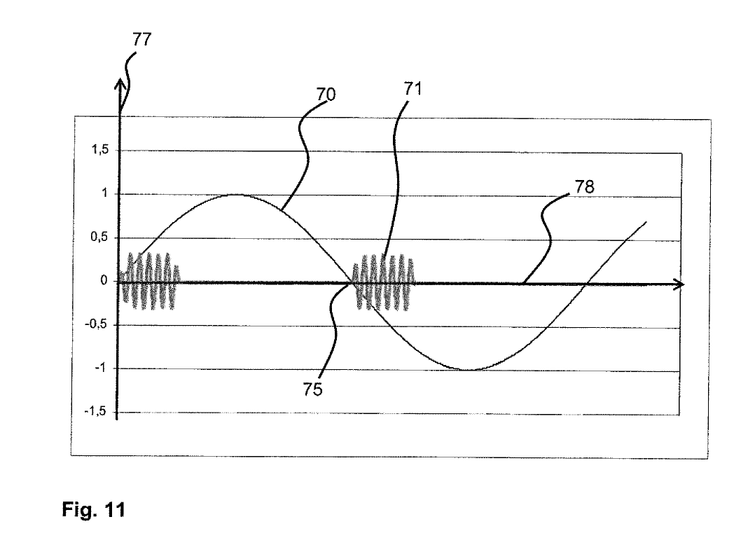

FIG. 11 schematically shows a second signal-vs.-time diagram with a view of EMG/MMG signal 72 and a breathing signal 70. The second signal-vs.-time diagram is obtained from the first signal-vs.-time diagram according to FIG. 10 by scaling the signal amplitude on the Y axis in a range of values 77 from −1.5 mV to +1.5 mV and by having suppressed the signal components of the ECG by filtering. The signal amplitude of the EMG/MMG signal 72 is typically lower than the ECG signal 71 (FIG. 10) by a factor of 10 to 5. The breathing signal 70 corresponds to the dimensionless and standardized breathing signal 70 in FIG. 10. The time interval on the time axis 78 corresponds to the time interval shown in FIG. 10. Reproducible signal patterns of the EMG/MMG signal 72 are seen at the changeover times 75 between inspiration and expiration. The EMG/MMG signal 72 yields evaluable and representative signal components at the beginning of inspiration and expiration.

On the whole, considerable advantages are associated with the medical sensor device 11 and with the medical sensor system 23. The sensor device 11 is divided into a disposable electrode 12 and a reusable holding element 10. As a result, only electrode 12 needs to be replaced after use and the holding element 10 can often be used over a rather long period of time. Due to the mechanomyographic sensors 17 being arranged at the reusable holding element 10, this advantageous and cost-effective division of the sensor device 11 into the disposable electrode 12 and the reusable holding element 10 can be maintained because the complicated and expensive mechanomyographic sensors 17 are arranged at the reusable holding element 10. Due to the proximity of the electrode 12 to the mechanomyographic sensors 17 in space and geometrically, it is possible to detect, in particular, the muscle efficiency and muscle fatigue with small artifacts between the mechanomyographic sensors 17 and electrode 12 as an electromyographic sensor.

While specific embodiments of the invention have been described in detail to illustrate the application of the principles of the invention, it will be understood that the invention may be embodied otherwise without departing from such principles.

What is claimed is:

1. A medical sensor device for a patient, the medical sensor device comprising:
an electrode for detecting an electric voltage on a body surface of the patient;
a holding element with a transmission means for transmitting or conducting signals or electric currents;
a detachable mechanical connection means for the detachable mechanical connection of the electrode with the holding element;
a detachable electric connection means for the detachable electric connection of the electrode with the holding element, the holding element comprising a sensor for detecting at least one medical parameter of the patient.

2. A medical sensor device in accordance with claim 1, wherein the sensor is a mechanomyographic sensor.

3. A medical sensor device in accordance with claim 1, wherein the sensor is an acceleration sensor and/or a microphone.

4. A medical sensor device in accordance with claim 1, wherein the detachable mechanical connection means has at least one elastic element for generating a prestress between the electrode and the holding element.

5. A medical sensor device in accordance with claim 1, wherein the detachable mechanical connection means provides a coefficient of friction between the holding element and the electrode that equals at least 0.05 to 0.25, including between a contact surface of the holding element and a contact surface of the electrode, at right angles to a contact surface of the electrode, and/or the prestressing force at the contact surface equals at least 0.5 N to 5 N.

6. A medical sensor device in accordance with claim 1, wherein the detachable mechanical connection provides static friction between the holding element and the electrode that equals 0.05 N to 10 N, including between the contact surface of holding element and the contact surface of the electrode, at right angles to contact surface of electrode.

7. A medical sensor device in accordance with claim 1, wherein the especially detachable mechanical connection means establishes at least one of a clip connection a locking connection, a frictionally engaged connection, a positive-locking connection and a nonpositive connection.

8. A medical sensor device in accordance with claim 1, wherein the detachable mechanical connection means comprises a mandrel formed at the electrode and a recess formed at the holding element, the mandrel being arranged in the recess.

9. A medical sensor device in accordance with claim 1, wherein the detachable mechanical connection means comprises an elastic element including a spring or an O-ring.

10. A medical sensor device in accordance with claim 1, wherein the electrode comprises a chamber and/or the contact surface including an adhesive surface portion.

11. A medical sensor device in accordance with claim 1, wherein the electrode is a disposable electrode and/or holding element that can be reused several times.

12. A medical sensor device in accordance with claim 1, wherein the transmission means for transmitting signals or electric currents comprises at least one of a cable, a signal processing and transmitting unit and a receiver.

13. A medical sensor system comprising:
a first medical sensor device comprising:
an electrode for detecting an electric voltage on a body surface of the patient;
a holding element comprising a sensor for detecting at least one medical parameter of the patient;
a detachable mechanical connection means for the detachable mechanical connection of the electrode with the holding element; and
a detachable electric connection means for the detachable electric connection of the electrode with the holding element;
a second medical sensor device comprising:
a second sensor electrode for detecting an electric voltage on a body surface of the patient;
a second sensor holding element;
a second sensor detachable mechanical connection means for the detachable mechanical connection of the second sensor electrode with the second sensor holding element; and
a second sensor detachable electric connection means for the detachable electric connection of the second sensor electrode with the second sensor holding element; and
transmission means connecting the a holding element to the second sensor holding element.

14. A medical sensor system according to claim 13, wherein:
the transmission means is at least one of a cable, a signal processing and transmitting unit and/or receiver; and
the sensor of the holding element for detecting at least one medical parameter of the patient is a mechanomyographic sensor arranged outside a holding portion of the holding element.
